(19) United States
(12) Patent Application Publication
(10) Pub. No.: US 2012/0190904 A1
(43) Pub. Date: Jul. 26, 2012

(54) HYDROGEN REMOVAL FROM DEHYDROGENATION REACTOR PRODUCT

(75) Inventor: James R. Butler, Spicewood, TX (US)

(73) Assignee: FINA TECHNOLOGY, INC., Houston, TX (US)

(21) Appl. No.: 13/352,339

(22) Filed: Jan. 18, 2012

Related U.S. Application Data
(60) Provisional application No. 61/434,546, filed on Jan. 20, 2011.

(51) Int. Cl. C07C 5/333; B01D 53/22; B01J 19/00

(52) U.S. Cl. 585/440; 585/654; 422/187; 95/55

(57) ABSTRACT

Disclosed is a dehydrogenation method that includes supplying a feed containing a hydrocarbon and steam into a dehydrogenation reactor containing a dehydrogenation catalyst, contacting the hydrocarbon and steam with the dehydrogenation catalyst to form a dehydrogenation product, wherein the dehydrogenation product comprises a dehydrogenated hydrocarbon, unreacted feed, steam and hydrogen, passing the dehydrogenation product through a membrane separator, and permeating hydrogen through a membrane positioned in the membrane separator. The hydrocarbon can be an alkyl aromatic and the dehydrogenated hydrocarbon can be a vinyl aromatic hydrocarbon, optionally the hydrocarbon can be an alkane and the dehydrogenated hydrocarbon can be an alkene.

### CROSS-REFERENCE TO RELATED APPLICATIONS

This application is a non-provisional of U.S. Provisional Application Ser. No. 61/434,546 filed Jan. 20, 2011.

### FIELD

Embodiments of the disclosed invention generally relate to dehydrogenation systems. In particular, embodiments of the disclosed invention relate to selective removal of hydrogen from dehydrogenation systems.

### BACKGROUND

Alkyl aromatic hydrocarbons are generally dehydrogenated by passing an alkyl-aromatic containing feed through a reactor containing a dehydrogenation catalyst. Steam may be mixed with the alkyl aromatic feed before the feed is introduced to the reactor and contacted with the dehydrogenation catalyst. The steam serves as a heat source to drive the endothermic reaction between the alkyl aromatic feed and the dehydrogenation catalyst so as to promote dehydrogenation. Additionally, the steam may inhibit the formation and deposition on the dehydrogenation catalyst of carbonaceous residues during dehydrogenation, thereby prolonging the useful life of the dehydrogenation catalyst.

After dehydrogenation of the alkyl-aromatic containing feed, the product contains a vinyl aromatic hydrocarbon, hydrogen, steam, and various other hydrocarbons (including unreacted feed). Energy may be recovered by condensing the product; however, such condensation can require a large condenser due to the low driving force for the heat exchange. One contributing factor to the large size of the condenser is the large amount of hydrogen present in the product. Hydrogen may lower the vapor pressure of the product, reduce the amount of heat transfer during condensation, and decrease the size of the compressors needed to pressure the feed to the dehydrogenation reactor.

Therefore, it is desirable to develop alternative methods of energy recovery in dehydrogenation processes.

### SUMMARY

An embodiment of the present invention, either by itself or in combination with other embodiments, includes supplying a feed containing a hydrocarbon and steam into a dehydrogenation reactor containing a dehydrogenation catalyst, contacting the hydrocarbon and steam with the dehydrogenation catalyst to form a dehydrogenation product, wherein the dehydrogenation product comprises a dehydrogenated hydrocarbon, unreacted feed, steam and hydrogen, passing the dehydrogenation product through a membrane separator, and permeating the hydrogen through a membrane positioned in the membrane separator.

The method can further include producing a retentate with the membrane, wherein the retentate includes the dehydrogenated and the unreacted feed. Optionally the retentate can include steam, and can have an increased dew point in comparison to the dehydrogenation product.

The hydrocarbon can be an alkyl aromatic hydrocarbon that can include ethylbenzene. Optionally the hydrocarbon can be an alkane.

The method can also include flowing the retentate to an azeotropic vaporizer and can further include desuperheating the dehydrogenation product to between 400 and 150° C., optionally to about 300° C.

An embodiment of the present invention for removal of hydrogen from a dehydrogenation product, either by itself or in combination with other embodiments, includes feeding the dehydrogenation product into a membrane separator, wherein the dehydrogenation product comprises hydrogen and steam, a dehydrogenated hydrocarbon, and an unreacted hydrocarbon, contacting the dehydrogenation product with a hydrogen selective membrane positioned in the membrane separator, permeating hydrogen through the hydrogen selective membrane, and flowing a retentate of the membrane separator to an azeotropic vaporizer.

The method can include desuperheating the dehydrogenation product, such as to between 400 and 150° C., optionally to about 300° C.

The retentate can include the dehydrogenated hydrocarbon, unreacted hydrocarbon, and steam.

The dehydrogenated hydrocarbon can be a vinyl aromatic hydrocarbon that can include styrene, wherein the unreacted hydrocarbon can be an alkyl aromatic hydrocarbon that can include ethylbenzene. The dehydrogenated hydrocarbon can be an alkene and the unreacted hydrocarbon can be an alkane.

The retentate can have a dew point above a vaporization temperature of an azeotrope of the unreacted alkyl aromatic hydrocarbon and water.

The hydrogen selective membrane can include a porous material, which can optionally be permeable only to hydrogen in the dehydrogenation product, or optionally can be an active transport membrane such as a Pd membrane.

An embodiment of the present invention, either by itself or in combination with other embodiments, includes a dehydrogenation system for dehydrogenating an alkyl aromatic hydrocarbon to a vinyl aromatic hydrocarbon, or optionally may dehydrogenate an alkane to form an alkene. The system including a dehydrogenation reactor containing a dehydrogenation catalyst, a membrane separator fluidly connected to the dehydrogenation reactor, and an azeotropic vaporizer fluidly connected to the membrane separator, wherein the membrane separator is positioned downstream of the dehydrogenation reactor, wherein the membrane separator is positioned upstream of the azeotropic vaporizer.

The dehydrogenation system can include a desuperheater fluidly connected to the dehydrogenation reactor and to the membrane separator, wherein the membrane separator is positioned downstream of the desuperheater.

The desuperheater can cool a dehydrogenation product of the dehydrogenation reactor to between 400 and 150° C.

The dehydrogenation system can also include a compressor fluidly connected to the azeotropic vaporizer and to the dehydrogenation reactor.

The dehydrogenation system membrane separator can include a membrane housing and a membrane positioned within the membrane housing, wherein the membrane is permeable to hydrogen. The membrane can be a low temperature material, that can optionally operate between 500° C. and 150° C.

### BRIEF DESCRIPTION OF DRAWINGS

FIG. 1 illustrates a flow diagram of embodiments of a method and system for hydrogen removal from a dehydrogenation reactor product.

### DETAILED DESCRIPTION

### Introduction and Definitions

The disclosed inventions generally include membrane separation of dehydrogenation products. For example, a membrane separator may be placed downstream of a reaction zone containing dehydrogenation reactors and upstream of an azeotropic vaporizer, for example.

Each of the inventions will now be described in detail below, including specific embodiments, versions and examples, but the inventions are not limited to these embodiments, versions or examples, which are included to enable a person having ordinary skill in the art to make and use the inventions when the information in this patent is combined with available information and technology. Each of the appended claims defines a separate invention, which for infringement purposes is recognized as including equivalents to the various elements or limitations specified in the claims.

Depending on the context, all references below to the term “invention” may in some cases refer to certain specific embodiments only. In other cases it will be recognized that references to the term “invention” will refer to subject matter recited in one or more, but not necessarily all, of the claims.

Various other terms as used herein are shown below. To the extent a term used in a claim is not defined below, it should be given the broadest definition skilled persons in the pertinent art have given that term as reflected in printed publications and issued patents at the time of filing. Further, unless otherwise specified, all compounds described herein may be substituted or unsubstituted and the listing of compounds includes derivatives thereof. Further, various ranges and/or numerical limitations may be expressly stated below. It should be recognized that unless stated otherwise, it is intended that endpoints are to be interchangeable. Further, any ranges include iterative ranges of like magnitude falling within the expressly stated ranges or limitations.

The term “dehydrogenation process(es)” may include contacting an alkyl aromatic hydrocarbon with a dehydrogenation catalyst to form a vinyl aromatic hydrocarbon within a dehydrogenation reactor, optionally may include contacting an alkane with a dehydrogenation catalyst to form an alkene within a dehydrogenation reactor.

The term “alkyl aromatic hydrocarbon” may include any alkyl aromatic hydrocarbon known to one skilled in the art, such as ethylbenzene, isopropylbenzene or ethyltoluene, for example.

The term “dehydrogenation catalyst” may include those catalysts known to one skilled in the art to be useful for dehydrogenation reactions, such as a reducible oxide of vanadium, for example. The term “reducible oxide” may refer to an oxide which is reduced by contact with hydrocarbons when operating under dehydrogenation conditions.

The term “dehydrogenation catalyst” may further include one or more promoters, such as alkali or alkaline-earth metals, for example. The term “dehydrogenation catalyst” may further include as non-limiting examples metal oxides, such as CuO, ZnO—CuO, ZnO—CuO—Al₂O₃; CuCr₂O₃; ZnCr₂O₃, ZnO—CuO—Cr₂O₃, or metals, such as Ru, Rh, Ni, Co, Pd or Pt supported on a substrate such as silica or titania, for example. The term “dehydrogenation catalyst” may further include a catalyst which may be prepared by methods known to one skilled in the art, such as absorption, precipitation, impregnation or combinations thereof, for example. See, U.S. Pat. No. 5,510,553, which is fully incorporated by reference herein. The term “dehydrogenation catalyst” may further include a catalyst that can be optionally bound to, supported on, or extruded with any suitable support material.

While described herein as a “dehydrogenation catalyst”, it is known to one skilled in the art that the term “catalyst” as used herein may refer to a compound that participates in the dehydrogenation reaction in addition to enhancing the rate of formation of the vinyl aromatic hydrocarbon.

The term “support material” may include oxides of metals, such as titanium, zirconium, zinc, magnesium, thorium, silica, calcium, barium and aluminum, clays and zeolitic materials, such as metallo-silicates or metallo-alumino-phosphates (e.g., alumino-silicates, borosilicates, silico-alumino-phosphates), for example. In one specific, non-limiting, embodiment, the dehydrogenation catalyst includes a reducible vanadium oxide on a magnesium oxide support.

The term “vinyl aromatic hydrocarbon”, which is formed via the methods described herein, is generally dependent upon the alkyl aromatic hydrocarbon and may include styrene, α-methyl styrene or vinyl toluene, for example.

The term “high temperature” refers to operating temperatures, such as reaction vessel and/or process stream temperatures of from about 150° C. to about 1000° C., or from about 300° C. to about 800° C., or from about 500° C. to about 700° C. or from about 550° C. to about 650° C., for example.

The term “low temperature” describes operating temperatures in the context of the membrane materials, and in particular, ceramic materials or Pd supported on ceramic materials used as membranes and is further defined in the context of each specific applications.

The term “membrane” may include a single membrane or multiple membranes configured in series, parallel or a combination of both, depending on the required hydrogen removal or other process conditions, for example.

The term “azeotrope” refers to a mixture of two or more liquids in such a ratio that its composition cannot be changed by simple distillation.

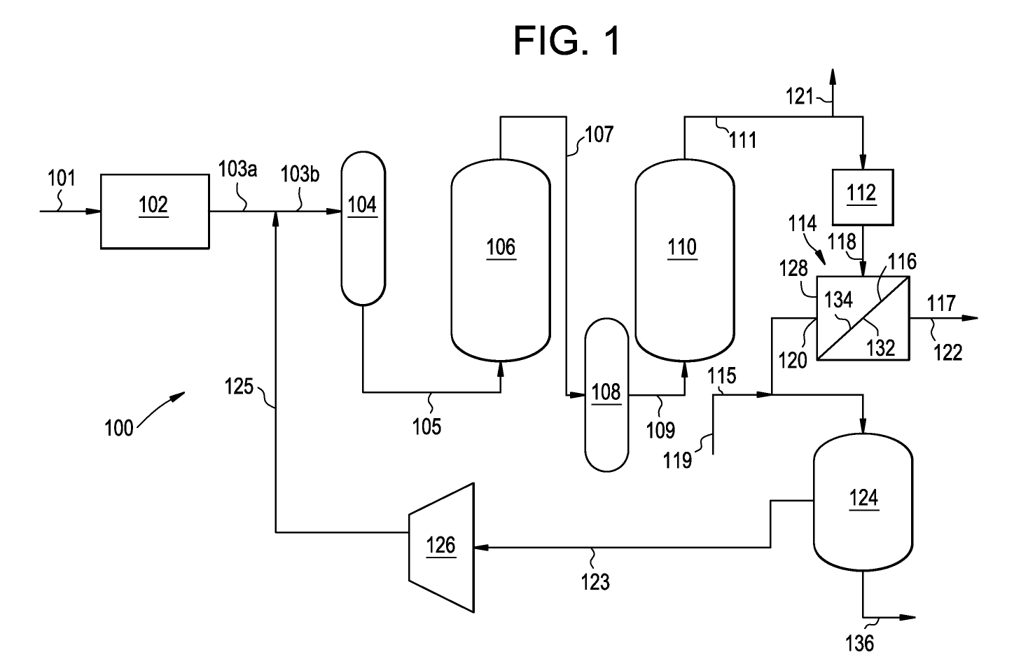

FIG. 1 illustrates a flow diagram of a specific, non-limiting, embodiment of a dehydrogenation system 100. The system 100 includes a membrane 116 used to remove hydrogen from a dehydrogenation product. In one or more embodiments, the system 100 may have dehydrogenation reactors 106 and 110 containing dehydrogenation catalysts, a membrane separator 114 fluidly connected to the dehydrogenation reactor 110, and an azeotropic vaporizer 124 fluidly connected to the membrane separator 114. The membrane separator 114 may be positioned downstream of the dehydrogenation reactors 106 and 110 and upstream of the azeotropic vaporizer 124. The system 100 may have a desuperheater 112 fluidly connected to dehydrogenation reactor 110 and to the membrane separator 114, and a compressor 126 fluidly connected to the azeotropic vaporizer 124 and to the dehydrogenation reactors 106 and 110. The membrane separator may be positioned downstream of the desuperheater 112. The compressor 126 may be positioned downstream of the azeotropic vaporizer 124 and upstream of the dehydrogenation reactors 106 and 110 (in a recycle loop context).

In one or more embodiments, the dehydrogenation system 100 shown in FIG. 1 may have a steam feed stream 101 and a hydrocarbon feed stream 119. The hydrocarbon undergoing dehydrogenation in the system 100 may be an alkyl aromatic such as ethylbenzene (EB), and the EB may be dehydrogenated to styrene, as is discussed by example hereinbelow. Optionally the hydrocarbon undergoing dehydrogenation in the system 100 may be an alkane such as by non-limiting example, butane or n-pentene, that can be dehydrogenated to alkenes such as butadiene or n-pentadiene.

The following example of EB dehydrogenation to styrene is presented as an example to illustrate the system and is not intended to be limiting. The feed stream 101 may flow into a steam superheater 102. Superheated steam may flow from the steam superheater 102 in process stream 103a to process stream 103b, where the superheated steam may mix with fresh/unreacted EB and flowing from process stream 125. Superheated steam, and fresh/unreacted EB may flow in process stream 103b to a first direct reheat unit 104. Reheated steam and EB may flow from the first direct reheat unit 104 to the first dehydrogenation reactor 106 via stream 105. The EB and steam may contact the dehydrogenation catalyst in the first dehydrogenation reactor 106 so as to dehydrogenate EB to styrene.

Hydrogen, styrene, and any unreacted EB may flow in process stream 107 from the first dehydrogenation reactor 106 to a direct reheat unit 108. After reheat, hydrogen, styrene, and any unreacted EB may flow in process stream 109 from the direct reheat unit 108 to a second dehydrogenation reactor 110. The unreacted EB may contact the dehydrogenation catalyst in the second dehydrogenation reactor 110 so as to dehydrogenate EB to styrene and produce hydrogen.

The dehydrogenation of EB creates a dehydrogenation product. The dehydrogenation product may contain hydrogen, styrene, and unreacted EB. The dehydrogenation product may flow in process stream 111 from the second dehydrogenation reactor 110 to a desuperheater 112. Styrene, the vinyl aromatic hydrocarbon, may flow in process stream 121 and can be recovered for further uses. In one or more embodiments, the desuperheater 112 may cool the dehydrogenation product to a temperature of from about 150° C. to about 400° C. or from about 175° C. to about 225° C. or from about 190° C. to about 210° C., for example. Desuperheated dehydrogenation product may flow in process stream 113 from the desuperheater 112 to a membrane separator 114.

In FIG. 1, the dehydrogenation product may contact a membrane 116 in the membrane separator 114, and hydrogen may permeate through the membrane 116 and may flow from the membrane separator 114 in process stream 117. The dehydrogenation product may flow in the membrane separator 114 to produce a retentate. The retentate may contain styrene, unreacted EB, steam and reduced amounts of hydrogen (in the event hydrogen is not completely removed), for example. The retentate may have a dew point that is above the vaporization temperature of an azeotrope of ethylbenzene and water. The retentate may flow in process stream 115 from the membrane separator 114 to the azeotropic vaporizer 124.

In FIG. 1, the azeotropic vaporizer 124 has a condensation side and a vaporization side. The condensation side condenses styrene, EB, and water that can exit via stream 136. The vaporization side vaporizes the ethylbenzene and water. The azeotropic vaporizer 124 may have a shell-and-tube type of heat-exchanger configuration, and the azeotropic vaporizer 124 may be similar to the vaporizer described in U.S. Pat. No. 4,695,664, which is herein incorporated by reference. The tube side may recover the heat of vaporization of the EB and water, conserving energy in the system 100.

EB from feed stream 119 may flow in process stream 123 from the azeotropic vaporizer 124 to a compressor 126. The compressor 126 may be one or more compressors in series or parallel or a combination thereof. The mole ratio of EB to steam in process stream 123 may be from about 0.5 to about 5, or from about 1 to about 4 or from about 1 to about 2.9. In one or more specific embodiments, the mole ratio of EB to steam is about 2.9. EB and steam may be compressed in the compressor 126. Compressed EB and steam may flow in process stream 125 from the compressor 126 to process stream 103b.

In one or more embodiments of the dehydrogenation system 100, the membrane separator 114 may be positioned downstream the second dehydrogenation reactor 110 and upstream of the azeotropic vaporizer 124. Regardless of the number of dehydrogenation reactors in a particular embodiment, the membrane separator 114 may be downstream of all dehydrogenation reactors. In an embodiment of the dehydrogenation system 100, the membrane separator 114 may be downstream of the desuperheater 112 and dehydrogenation reactors 106 and 110. Placement of the membrane separator 114 downstream of all dehydrogenation reactors 106 and 110 and upstream of the azeotropic vaporizer 124 allows removal of hydrogen before the dehydrogenation product flows to the azeotropic vaporizer 124, which results in the advantages discussed below.

It is further contemplated that the system 100 may have only one dehydrogenation reactor or more than two dehydrogenation reactors. Embodiments of the dehydrogenation reactor 106 and 110 may include, by non-limiting examples: fixed bed reactors; fluid bed reactors; and entrained bed reactors. Reactors capable of the elevated temperature and pressure as described herein, and capable of enabling contact of the reactants with the catalyst, can be considered within the scope of the disclosure. Embodiments of the dehydrogenation reactors 106 and 110 may be determined based on the particular design conditions and throughput, as by one of ordinary skill in the art, and are not meant to be limiting on the scope of the disclosure. The dehydrogenation reactor(s) may be of various configurations including a radial flow reactor such as disclosed in U.S. Pat. No. 5,358,698 or a linear or tubular reactor such as disclosed in U.S. Pat. Nos. 4,287,375 and 4,549,032, all of which are incorporated by reference herein.

To aid in the flow of the applicable materials, such as steam, feed and catalyst, the dehydrogenation system 100 generally utilizes a pressure drop. The applicable materials generally have a short residence time in the reaction zone of the dehydrogenation reactors 106 and 110, further aiding in maintaining the oxidation state in the desired tolerances. For example, the feed may have a residence time of from about 0.005 seconds to about 30 seconds or from about 0.010 seconds to about 15 seconds and the catalyst may have a residence time of from about 0.5 seconds to about 5 minutes or from about 1 second to about 1 minute.

As discussed previously herein, it is contemplated that the alkyl aromatic hydrocarbon, e.g., EB in FIG. 1, may contact the dehydrogenation catalyst in the presence of an inert diluent, such as steam. Such contact may occur in any manner known to one skilled in the art. For example, the diluent may be added to the alkyl aromatic hydrocarbon prior to contact with the catalyst, for example. Although the amount of diluent contacting the alkyl aromatic hydrocarbon is determined by individual process parameters, the diluent may contact the alkyl aromatic hydrocarbon in a weight ratio of from about 0.01:1 to about 15:1, or from about 0.3:1 to about 10:1, or from about 0.6:1 to about 3:1 or from about 1:1 to about 2:1, for example.

It is further contemplated that the dehydrogenation system 100 may include a single or a plurality of stages. When utilizing a plurality of stages, such stages may be housed in a single reaction vessel, or in multiple reaction vessels, for example. In one or more embodiments, the multiple reaction vessels include series connected dehydrogenation reactions.

The presence of hydrogen (often at significant levels) in the first dehydrogenation reactor 106 and second dehydrogenation reactor 110 often requires significant inert diluents/steam feed rates in order to overcome equilibrium limits on conversion. For example, the dehydrogenation processes may include a steam to alkyl aromatic hydrocarbon feed ratio of from about 5 to about 25, or from about 8 to about 20 of from about 10 to about 15, for example. However, selective hydrogen removal from the dehydrogenation system 100 lowers the level of steam required to overcome equilibrium limits on conversion, thereby resulting in energy savings of the system.

Dehydrogenation product may flow from process stream 113 to the inlet 118 of the membrane housing 128 of the membrane separator 114. The membrane 116 may have a permeate side 132 and a retentate side 134. Hydrogen may permeate through the permeate side 132 of the membrane 116, and the dehydrogenation product may flow on the retentate side 134 of the membrane 116 to form the retentate, which may flow out of the membrane housing 128 through the retentate outlet 120.

In one or more embodiments, the membrane 116 may include a hydrogen permeable membrane. Permeated hydrogen may flow on the permeate side 132 of the membrane 116 out of the membrane housing 128 through the permeate outlet 122. The hydrogen permeable membrane 116 may be formed of any material which exhibits substantial permeability to hydrogen while being substantially impermeable to the larger molecules involved in the dehydrogenation reaction, such as an inert diluent (e.g., steam in FIG. 1), an alkyl aromatic hydrocarbon (e.g., EB in FIG. 1), and a vinyl aromatic hydrocarbon (e.g., styrene in FIG. 1), for example.

In one or more embodiments, the membrane 116 is formed of a ceramic material.

In one or more embodiments, the membrane 116 is an inorganic membrane.

In one or more embodiments, the membrane 116 is made of a porous material.

In one or more embodiments, the membrane 116 is formed of a sintered metal, such as palladium, copper, alloys thereof and combinations thereof, for example. In one or more specific embodiments, the membrane 116 is formed of palladium and from about 35 wt. % to about 45 wt. % copper, for example.

In one or more embodiments, the membrane 116 may remove hydrogen from the dehydrogenation product via physical separation, e.g., ultrafiltration or nanofiltration. It is contemplated that the membrane 116 may remove hydrogen from the dehydrogenation product via other pathways such as electrochemical separation or a combination of physical and electrochemical separation.

In one or more embodiments, the membrane 116 may be supported on a thin film, monolith, disc, or the like, for example.

In one or more embodiments, the retentate may contain vinyl aromatic hydrocarbon and unreacted feed. Alternatively, the retentate may contain vinyl aromatic hydrocarbon and unreacted alkyl aromatic hydrocarbon. The unreacted alkyl aromatic hydrocarbon may be EB. Optionally the retentate may contain alkene and unreacted feed that includes alkanes.

In one or more embodiments, the rententate may have a dew point that is above the dew point of the reactor effluent. In FIG. 1, the rententate may have a dew point that is above the vaporization temperature of an azeotrope of ethylbenzene and water.

The process stream flow shown in FIG. 1 may be modified based on unit optimization so long as the modification complies with the spirit of the invention, as defined by the claims. For example, additional process equipment, such as additional dehydrogenation reactors or membrane separators, may be employed throughout the methods 100 described herein and such placement is generally known to one skilled in the art. Further, while described below in terms of primary components, the streams indicated below may include any additional components as known to one skilled in the art.

The embodiments described herein result in the ability to remove hydrogen with the membrane separator 114 from the dehydrogenation product before the dehydrogenation product is sent to the azeotropic vaporizer 124 and after the dehydrogenation product has passed the reaction zone containing any dehydrogenation reactors (e.g., 106 and 110 in FIG. 1). As a result, several advantages occur.

The vapor pressure of the retentate is higher than the vapor pressure of the dehydrogenation product. The retentate flowing in process stream 115 from the membrane separator 114 to the azeotropic vaporizer 124 has a higher vapor pressure than if hydrogen were not removed by the membrane separator 114.

Removing hydrogen before the dehydrogenation product flows to the azeotropic vaporizer 124 decreases the vapor pressure of the retentate in the azeotropic vaporizer 124, and thus facilitates its condensation in the azeotropic vaporizer 124 making it easier to recover the heat of condensation. This energy can then be used to vaporize or heat the feed, such as ethylbenzene and/or steam going to the reactor. Removal of hydrogen in the membrane separator 114 thus creates an efficiency of heat transfer in the retentate that is greater than the efficiency of heat transfer of the dehydrogenation product. The greater efficiency of heat transfer of the retentate can reduce the size of the equipment of the azeotropic vaporizer 124 that is needed in order to condense the product containing reduced amounts of hydrogen.

Removal of hydrogen in the membrane separator 114 can increase the dew point of the remaining components found in the retentate above the vaporization temperature of the azeotrope for alkyl aromatic compound and water. The higher dew point can reduce the cost of azeotropic vaporization and reduce the amount of steam needed for the process.

Removal of hydrogen in the membrane separator 114 can increase the dew point of the condensed liquids in the azeotropic vaporizer 124, making recovery of the heat of the vaporization of the alkyl aromatic hydrocarbon and water azeotrope easier.

Removal of hydrogen in the membrane separator 114 can reduce the size of the compressor 126 which pressures the feed in process stream 115 to the dehydrogenation reactors 106 and 110 in process stream 125. With reduced amounts of hydrogen in process streams 115 and 123, any compression required is less than if hydrogen were not removed in the membrane separator 114. Less compression reduces the size of the compressor(s) needed and may reduce the need for compressors altogether. Given that compression is a significant cost in the processes described herein, reductions in compression resulting from removal of hydrogen in the membrane separator result in significant cost savings.

The various aspects of the present invention can be joined in combination with other aspects of the invention and the listed embodiments herein are not meant to limit the invention. All combinations of various aspects of the invention are enabled, even if not given in a particular example herein.

While illustrative embodiments have been depicted and described, modifications thereof can be made by one skilled in the art without departing from the spirit and scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the aspects and embodiments disclosed herein are usable and combinable with every other embodiment and/or aspect disclosed herein, and consequently, this disclosure is enabling for any and all combinations of the embodiments and/or aspects disclosed herein. Other and further embodiments, versions and examples of the invention may be devised without departing from the basic scope thereof and the scope thereof is determined by the claims that follow.

While the foregoing is directed to embodiments of the present invention, other and further embodiments of the invention may be devised without departing from the basic scope thereof and the scope thereof is determined by the claims that follow.

What is claimed is:

1. A method comprising:
supplying a feed containing a hydrocarbon and steam into a dehydrogenation reactor containing a dehydrogenation catalyst;
contacting the hydrocarbon and steam with the dehydrogenation catalyst to form a dehydrogenation product, wherein the dehydrogenation product comprises a dehydrogenated hydrocarbon, unreacted feed, steam and hydrogen;
passing the dehydrogenation product through a membrane separator; and
permeating the hydrogen through a membrane positioned in the membrane separator.

2. The method of claim 1, further comprising:
producing a retentate with the membrane, wherein the retentate comprises dehydrogenation product and unreacted feed.

3. The method of claim 1, wherein the hydrocarbon comprises an alkyl aromatic hydrocarbon and the dehydrogenation product comprises a vinyl aromatic hydrocarbon.

4. The method of claim 1, wherein the hydrocarbon comprises an alkane and the dehydrogenation product comprises an alkene.

5. The method of claim 2, wherein the retentate further comprises steam.

6. The method of claim 2, wherein the retentate has an increased dew point in comparison to the dehydrogenation product.

7. The method of claim 3, wherein the alkyl aromatic hydrocarbon comprises ethylbenzene.

8. The method of claim 2, further comprising flowing the retentate to an azeotropic vaporizer.

9. The method of claim 1, further comprising:
desuperheating the dehydrogenation product to from 400° C. to 150° C.

10. The method of claim 1, wherein the membrane separator comprises a hydrogen permeable porous material.

11. The method of claim 10, wherein the porous material is permeable only to hydrogen in the dehydrogenation product.

12. A method for removal of hydrogen from a dehydrogenation product comprising:
feeding a dehydrogenation product into a membrane separator, wherein the dehydrogenation product comprises hydrogen and steam, a dehydrogenated hydrocarbon, and an unreacted hydrocarbon;
contacting the dehydrogenation product with a hydrogen permeable membrane positioned in the membrane separator;
permeating hydrogen through the hydrogen permeable membrane; and
flowing a retentate of the membrane separator to an azeotropic vaporizer.

13. The method of claim 12, further comprising:
desuperheating the dehydrogenation product to from 400° C. to 150° C.

14. The method of claim 12, wherein the retentate comprises vinyl aromatic hydrocarbon, unreacted alkyl aromatic hydrocarbon, and steam.

15. The method of claim 14, wherein the vinyl aromatic hydrocarbon comprises styrene, wherein the unreacted alkyl aromatic hydrocarbon comprises ethylbenzene.

16. The method of claim 12, wherein the retentate has a dew point nearer a vaporization temperature of an azeotrope of the unreacted hydrocarbon and water.

17. The method of claim 12, wherein the hydrogen permeable membrane comprises a porous material.

18. The method of claim 17, wherein the porous material is permeable only to hydrogen in the dehydrogenation product.

19. The method of claim 12, wherein the unreacted hydrocarbon comprises an alkane and the dehydrogenated hydrocarbon comprises an alkene.

20. A dehydrogenation system for dehydrogenating a hydrocarbon to a dehydrogenated hydrocarbon comprising:
a dehydrogenation reactor containing a dehydrogenation catalyst;
a membrane separator fluidly connected to the dehydrogenation reactor; and
an azeotropic vaporizer fluidly connected to the membrane separator, wherein the membrane separator is positioned downstream of the dehydrogenation reactor, wherein the membrane separator is positioned upstream of the azeotropic vaporizer.

21. The dehydrogenation system of claim 20, further comprising:
a desuperheater fluidly connected to the dehydrogenation reactor and to the membrane separator, wherein the membrane separator is positioned downstream of the desuperheater.

22. The dehydrogenation system of claim 21, wherein the desuperheater cools a dehydrogenation product of the dehydrogenation reactor to from 400° C. to 150° C.

23. The dehydrogenation system of claim 20, further comprising:
a compressor fluidly connected to the azeotropic vaporizer and to the dehydrogenation reactor.

24. The dehydrogenation system of claim 20, wherein the membrane separator comprises:
a membrane housing; and
a membrane positioned within the membrane housing, wherein the membrane is permeable to hydrogen.

25. The dehydrogenation system of claim 24, wherein the membrane comprises a low temperature porous material.

26. The dehydrogenation system of claim 25, wherein the membrane operates between 500° C. and 150° C.

27. The dehydrogenation system of claim 20, wherein the hydrocarbon is an alkyl aromatic hydrocarbon and the dehydrogenated hydrocarbon is a vinyl aromatic hydrocarbon.

28. The dehydrogenation system of claim 20, wherein the unreacted hydrocarbon comprises an alkane and the dehydrogenated hydrocarbon comprises an alkene.
